(19) United States
(12) Patent Application Publication
(10) Pub. No.: US 2013/0325082 A1
(43) Pub. Date: Dec. 5, 2013

(54) APPARATUS THAT BROADCASTS SPECIFIC BRAIN WAVEFORMS TO MODULATE BODY ORGAN FUNCTIONING

(71) Applicant: Eleanor Schuler, Rio Rancho, NM (US)

(72) Inventor: Eleanor Schuler, Rio Rancho, NM (US)

(21) Appl. No.: 13/932,295

(22) Filed: Jul. 1, 2013

Related U.S. Application Data
(63) Continuation-in-part of application No. 11/437,096, filed on May 19, 2006, now Pat. No. 8,509,887.
Application No. 11/437,096 is a continuation of application No. 10/000,005, filed on Nov. 20, 2001, now Pat. No. 7,308,302.
(60) Provisional application No. 60/249,882, filed on Nov. 20, 2000.

(51) Int. Cl. A61N 1/36

(52) U.S. Cl. CPC A61N 1/36057
USPC 607/42; 607/62

(57) ABSTRACT

Apparatus for collecting and broadcasting coded human or animal body waveforms. A contact can be placed on a portion of a body, which is designed to receive electrical signals. The electrical signal is converted into a readable format and is processed and stored in a computer. The electrical signal can be adjusted and rebroadcast into the body to modulate body organ functioning including, for example, cardiovascular, respiratory, digestive, and other organ functioning.

### CROSS-REFERENCE TO RELATED APPLICATIONS

This patent application is a continuation of U.S. patent application Ser. No. 11/437,096, filed May 19, 2006, which is a continuation of U.S. patent application Ser. No. 10/000,005, filed Nov. 20, 2001, which claims the benefit of U.S. Provisional Application No. 60/249,882, filed Nov. 20, 2000. This patent application therefore traces its priority date to the Nov. 20, 2000 filing date of U.S. Provisional Application No. 60/249,882. U.S. patent application Ser. No. 11/437,096, U.S. patent application Ser. No. 10/000,005, and U.S. Provisional Application No. 60/249,882 are incorporated herein by reference in their entireties.

### FIELD OF THE INVENTION

Embodiments are generally related to coded electrical waveforms and methods for collecting and interpreting signals from the brain.

### BACKGROUND

The brain is one of the last great frontiers in the bio-medical sciences. The unraveling of its mysterious complexities as related to medical diagnosis and treatment is a quest as great as inventing technology and gathering resources to travel to the moon. Brain signals direct the harmony of the human body much like a conductor controls and directs his orchestra. The brain senses, computes and decides before it sends electrical and chemical instructions to the body it lives in. The brain is a magnificent information processor that not only controls the body it lives in, but communicates with other brains residing in other bodies. Such interrelation to another brain can alter the electrochemical function in both brains.

Like no other creature, mankind over the centuries has slowly observed his own health status and devised treatments to heal diseases and injuries. Because historically man has preserved this medical knowledge in books it served as the basis of early university scientific training. The last two centuries of education and research in biomedicine have laid down a detailed understanding about the human anatomy and the relative function of its components, all of which serve as a platform for today's medical treatments.

Modern scientists have expanded into specialties that never existed before. Today, scientists study the genetic makeup of humans and are heading toward predicting and tinkering with genes to forestall future ailments. Then there are studies on a cellular level that have determined the microscopic working of many of the ubiquitous chemical and electrical processes that link and regulate life processes.

Although scientists and physicians can treat every organ in the body with surgery or medications, it is only in the last half century that we have come to grips with electrical treatment of organ systems. Examples of this development are the cardiac defibrillator and pacemaker or electrical brain stimulator for Parkinson's. Meticulous anatomical studies, animal experiments and recording the consequences of human brain injuries and diseases have served as the base information to understand how the brain works.

There has been dynamic cellular and molecular biology work performed in university laboratories over the past 20 years that is still ongoing. This has opened up bio-functional details that were previously unknown, in addition, recent publications of marvelous texts on neuroanatomy and physiology have illuminated the physical relationship to actual function of the nervous system.

This fountain of knowledge now makes it possible to open up a new technology for electrical modulation of organ function. Such knowledge opens new electrical treatment modalities for life threatening emergencies and cardiac, respiratory and digestive conditions, inaccessible before. This new technology makes it possible to detect the electrical waveforms being generated by the brain and to ascertain what the signal is for. This invention provides a way to evolve the known and unknown waveforms into electronic devices, which can broadcast such signals onto selected nervous system components as medical treatments.

It is not commonly understood how brain electrical signals modulate functions of the body as a whole, but there is an understanding to a limited degree of how organs are modulated. The brain controls critical functions of all human and animal body organ systems in a coordinated way to keep the body alive and hence to keep alive the brain itself. The brain wants to live and go on into the future so it fine-tunes and modulates the cardiovascular, respiratory, and digestive systems among others to integrate the needs of all. Maintaining optimum performance is more difficult as the body and brain age due to cellular degradation. But if critical organ functions can be reset in a non- or minimally invasive way, both quality and life-extension may benefit.

The brain controls, via the autonomic nervous network, the vegetative functions of the major organs. These organs represent the minimal requirement to support life. These are the organs that must function even if the brain is in coma and the owner unable to think or do anything, if life is to continue. Major organ function must always be maintained at a certain minimal level for maintaining organism life, otherwise death is certain. Such control is done via a nervous system that consists of two main divisions: a) the central nervous system (brain) in concert with the spinal cord, and b) the peripheral system consisting of cranial and spinal nerves plus ganglia.

Within the central nervous system is the autonomic nervous system (ANS) which carries all efferent impulses except for the motor innervations of skeletal muscles. The ANS is mainly outside voluntary control and regulates the heartbeat and smooth muscle contraction of many organs including digestive and respiratory. Also, the ANS controls exocrine and some endocrine organs along with certain metabolic activity. In addition, there is activity from parasympathetic and sympathetic innervations, which oppose each other to attain a balance of tissue and organ function. The nervous system is constructed of nerve cells called neurons, which have supporting cells called glia. Neurons are electrically excitable and provide a method whereby instructions are carried from the brain to modulate critical functions.

The neuron has a protrusion called an axon that can be as short as a few millimeters or longer than a meter. The axon provides and uses nerve fibers to carry electrical signals that end at a synapse. A synapse is at the end of an axon. It faces another synapse from a neighboring axon across a gap. To cross such a gap the electrical signal from the brain must engage in specialized chemical or electrical transduction reactions to allow the crossing of the electrical signal to the next axon or to a nerve plexus or ganglion located on an actual organ. Neurons have a body (or soma) and are the morphological and functioning unit that sends signals along their axons until such signals instruct the organ it reaches. Operative neuron units that carry signals from the brain are classified as “efferent” nerves. “Afferent” nerves are those that carry sensor or status information to the brain. The brain computes and generates those electrical signals that are required as a result of the incoming data (afferent signals) it has collected. Such afferent signals received by the brain provide sophisticated organ and overall body operational status. Such information spans the entire body from within and also environmental status detected from areas immediately outside of the body proper and at some distance.

Outside data reaching the brain may relate to temperature change or a dangerous situation like approaching strangers or even potential mating possibilities. Eyes, ears, nose, tongue and skin provide such outside afferent sensory data. In addition, there is proprioception providing sensation in the musculoskeletal system, i.e., deep sensations. Other afferent-type nerve sensors called nociceptors detect noxious stimuli and pain. Nociceptors alert the brain to nasty things that are deemed undesirable and require some immediate action within the brain. This range of information arriving at the brain is processed for action. The efferent nerves provide quick adjustment on performance for the various organ systems or even systems or even instruct the skeletal-motor neurons to rim, walk, hide, help or physically approach for more sensory information.

The invention describes specific waveforms and a method to precisely acquire the key operative electrical waveforms from selected axons, nerveplexus or ganglion connections of the autonomic nervous system. Such waveform data is stored and categorized as to the actual purpose of such signals. This is much like the ongoing effort to identify and categorize human genes. Once the purpose of individual coded electrical waveforms have been determined, they will be installed in a specific application microprocessor for electrical broadcast or conduction into the nervous system, in order to treat or correct selected medical conditions.

### SUMMARY

The following summary is provided to facilitate an understanding of some of the innovative features unique to the disclosed embodiments and is not intended to be a full description. A full appreciation of the various aspects of the embodiments disclosed herein can be gained by taking the entire specification, claims, drawings, and abstract as a whole.

The invention provides a method for modulating body organ functioning. According to the method, waveforms that are generated and carried in a body are collected from the body. Such collected waveforms are then electrically stored. Then, one or more of the collected waveforms can be transmitted to a body organ to stimulate or regulate organ function.

The collected waveforms are transformed into a readable format for a processor. The transformation of the collected coded waveforms into a readable format includes transforming analog signals into a digital form. The collected waveforms are stored and cataloged according to the function performed by the waveforms in the body. A digital to analog converter is used to convert the cataloged waveforms to an analog form, and the converted waveforms are then applied to a body organ to regulate for medical treatment purposes.

The invention further provides an apparatus for modulating body organ functioning. The apparatus includes a source of collected waveforms that are indicative of body organ functioning, means for transmitting collected waveforms to a body organ, and means for applying the transmitted waveforms to the body organ to stimulate or adjust organ function.

The transmitting means may include a digital to analog converter. The source of collected waveforms comprises a computer which has the collected waveforms stored in digital format. The computer includes separate storage areas for collected waveforms of different categories.

The apparatus further includes means for collecting waveforms from a body and cataloging and transmitting such collected waveforms to the source. The collecting means may be comprised of a sensor placed on the body. A recorder is provided to record the sensed waveforms in analog form. An analog to digital converter is connected to the recorder for converting the waveforms before being sent to a scientific computer. Additionally, the apparatus includes a digital to analog converter for converting the collected waveforms for retransmission to a body for medical treatment purposes.

The aforementioned aspects and other objectives and advantages can now be achieved as described herein.

### BRIEF DESCRIPTION OF THE FIGURES

The accompanying figures, in which like reference numerals refer to identical or functionally-similar elements throughout the separate views and which are incorporated in and form a part of the specification, further illustrate the present invention and, together with the detailed description, serve to explain the principles of the present invention.

FIG. 1 is a schematic diagram of one form of apparatus for practicing the method according to an embodiment;

FIG. 2 illustrates a high-level flow chart of operations of a method capable of being implemented by a software program when the waveform enters the computer;

FIG. 3 illustrates a high-level flow chart of operations of a method capable of being implemented by a software program when the operator retrieves and broadcasts the waveform from within the computer;

FIGS. 4A-4H illustrate schematics of representative waveforms, embodied in the invention, carried by neurons after generation in the medulla oblongata or from sensory neurons going to the medulla oblongata;

FIGS. 5A-5G illustrate schematics of alternative waveforms, as described in the invention, that affect the nervous system;

FIG. 6 illustrates a schematic view of a computer system, which can be implemented in accordance with one or more of the disclosed embodiments; and

FIG. 7 illustrates a schematic view of a software system, an operating system, and a user interface, in accordance with one or more embodiments.

### DETAILED DESCRIPTION

The particular values and configurations discussed in these non-limiting examples can be varied and are cited merely to illustrate at least one embodiment and are not intended to limit the scope thereof.

For the purpose of promoting an understanding of the principles of the invention, references will be made to the embodiments illustrated in the drawings. It will, nevertheless, be understood that no limitation of the scope of the invention is thereby intended, such alterations and further modifications in the illustrated device, and such farther applications of the principles of the invention illustrated herein being contemplated as would normally occur to the one skilled in the art to which the invention relates.

Human and other mammals, and even lower creatures of all types, generate electrical wave-forms from their respective brains that modulate key aspects of vegetative systems. Such waveforms are of similar general linear analog format in appearance, regardless of species. Parallel lines of signals also can be transmitted simultaneously by the medulla oblongata to help form the signaling waveforms. Key organ systems such as cardiovascular, respiratory, digestive, and others decode these signals and modulate or fine-tune themselves in response to those instructions. The autonomic nervous system (ANS) operates similarly in all species, but not exactly similar. The parallel carriers of autonomic signals may work as the lines on a sheet of music record notes of different characteristic, pause or speed at different levels. The autonomic nervous system operates without willful or conscious control and generally control vegetative state essential body organ systems.

This invention focuses on the electrical signals transported by the vagus accessory and hypoglossal nerve bundles, including afferent fibers. The vagus nerve is a wandering nerve (Vagus means wandering) that winds throughout the body after it emerges from the medulla oblongata located in the hind brain. The hypoglossal and accessory nerves also emerge from the medulla oblongata and are interlaced with the vagus to harmoniously accomplish basic life support. The signals travel on the surface of the vagus nerve but below its insulating myelin sheath.

The electrical output of selected afferent and efferent nerves can be made accessible via silver, gold or other metal wires, or voltage clamps or patch electrodes and even seismic sensors, along with other detection methods. The particular apparatus for detecting this output is not part of the present invention. Afferent and efferent nerves travel in the same nerve bundles or can be routed separately. To gain direct measurement of the electrical waveforms, it may initially require shaving away the insulating fasciculus and myelin sheath. Seismic, ultrasonic, receiving antennas, direct conduction and other methods may be used to capture the coded brain signals as they relate to body organ performance. Such signals are then stored and replicated for electrical return to the appropriate place for medical treatment concerned with modulating organ function.

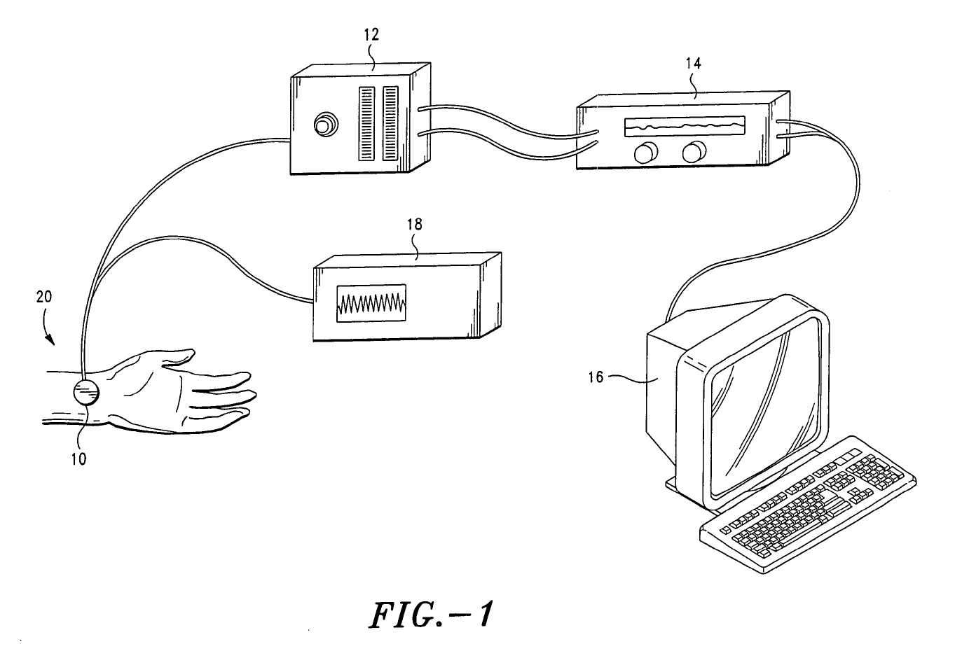

The invention comprises a method for recording, storing, and broadcasting specific brain waveforms to modulate human and animal body organ functioning. One form of the method for recording, storing, and broadcasting brain waveforms, as shown in FIG. 1, is comprised of at least one sensor in the form of an electrode or pair of electrodes 10, an analog recorder 12, and an analog to digital converter 14, a computer 16, and a digital to analog converter 18. The electrode 10 is attached to a nerve 20 in the human or animal body and receives the coded electric waveform form the nerve 20. The electrode 10 may be comprised of silver wire, tungsten wire, or any wire suitable for conduction of the perceptible electrical signals transported by the nerve 20.

An analog recorder 12 records the electric waveform because the nerve 20 only transmits electric signals in analog form. Once the waveforms are recorded they are sent from the analog recorder 12 to the analog to digital converter 14. The converter 14, in a conventional fashion, transforms the waveforms from the analog format into a digital format, which is more suitable for computer processing. The converter 14 then transmits the converted waveforms to a computer 16 where the waveform is processed, stored, adjusted, and/or broadcast, as desired.

Selected signals that have been digitized may be transferred to an application specific processor or a linear analog device to be utilized to prepare and broadcast signals recognized by the brain or a selected organ as a modulating treatment. When the operator directs the computer 16 to retrieve and broadcast the waveform back into the body, the waveform is transmitted from the computer 16 through a digital to analog converter 18. In a conventional fashion, the waveform is converted back into analog form because the body only transmits and uses coded electrical signals in analog format. If the coded waveforms were transmitted into the body in a digital form, the body would not recognize the transmission.

The computer 16 contains software, which is capable of identifying the function associated with particular waveforms. Many types of software can be developed by those skilled in the art to perform the functions of the invention, and particular software is not part of the present invention. As shown in the flow chart in FIG. 2, after beginning at step 22, at step 24 the computer 16 receives a digital waveform from the analog to digital converter 14. After the waveform is received, the software reads the waveform and at step 26 identifies the function of the particular waveform. Once the software identifies the function associated with the particular waveform, at step 28 the waveform or coded signal is directed to a particularized storage area. For example, if the waveform is used for digestive functions it may be stored in a separate area from waveforms used for respiratory functions.

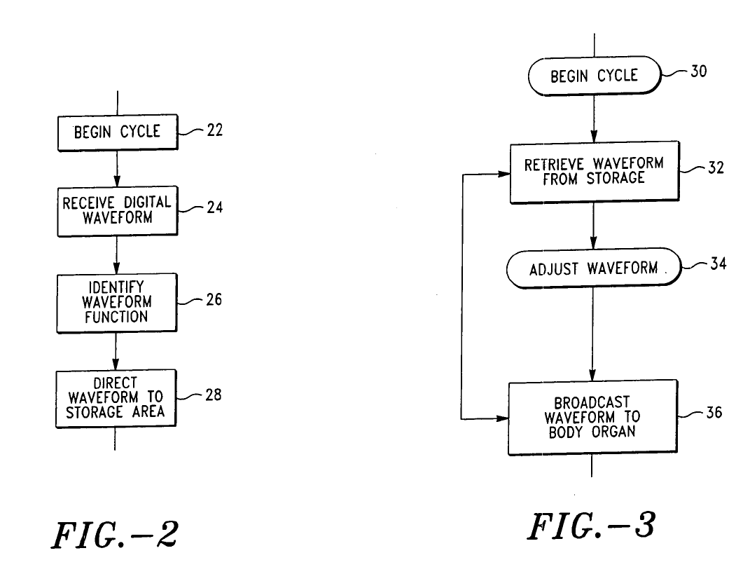

Later, when it is decided to use the stored digital form of the waveform, as shown in the flow chart in FIG. 3, the cycle is begun at 30, and the waveform is retrieved from the storage area, as shown at step 32, having been previously stored at step 28 (FIG. 2). If it is determined that the waveform needs to be adjusted in order to perform a particular function, the software adjusts the waveform as required, in step 34. However, if it is decided that the waveform does not need to be adjusted, step 34 is bypassed and step 36 is performed whereby the waveform signal is broadcast to the specified body organ, after conversion to analog form. The brain often makes modifications to the waveforms in order to fine-tune the function the brain requires or needs a particular organ to perform, and such is also performed by the present invention.

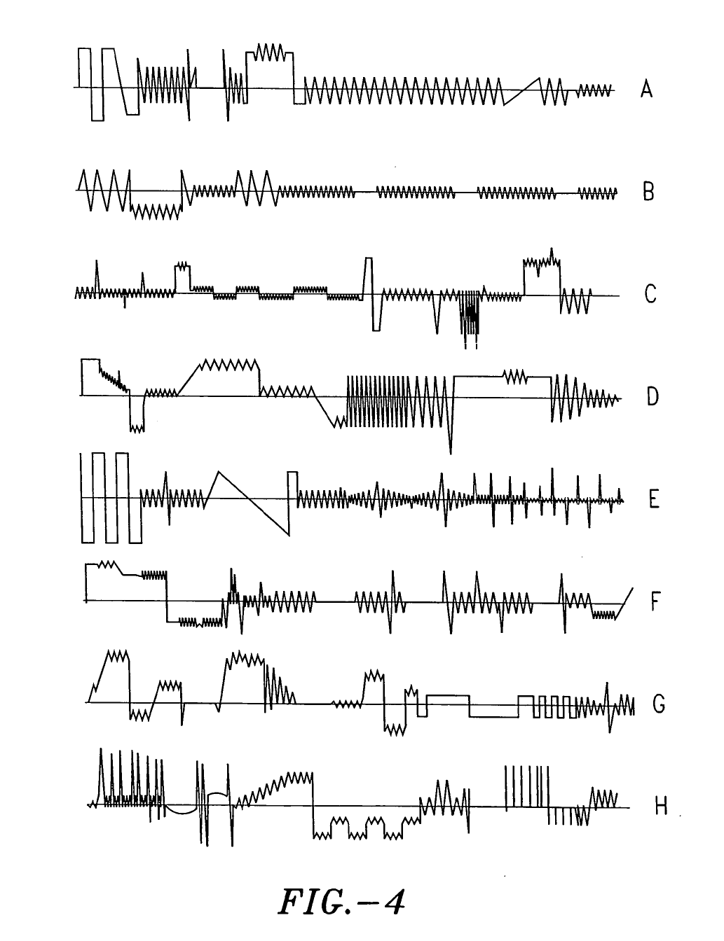

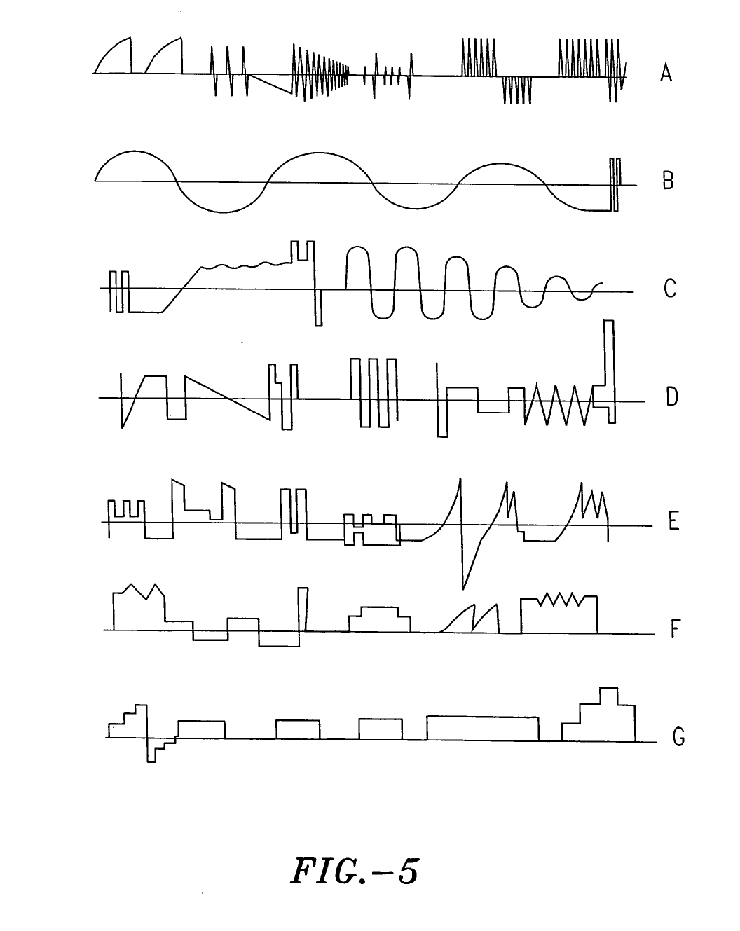

Representative waveforms that neurons carry after generation in the medulla oblongata are shown in FIG. 4. Such waveforms have a central linear carrier, which is analog. The signal is of a direct current nature and has many coded modulations that provide directions or instructions to the receptor organ or system receiving it. Other representative waveforms for signals that can affect the nervous system are shown in FIG. 5. The waveforms can provide instructions as they leave the vagus or other nerve and arrive at organs of the body. Such signals are similar to the modulating instructions broadcast from the medulla oblongata.

The embodiments are described at least in part herein with reference to flowchart illustrations and/or block diagrams of methods, systems, and computer program products and data structures according to embodiments of the invention. It will be understood that each block of the illustrations, and combinations of blocks, can be implemented by computer program instructions. These computer program instructions may be provided to a processor of a general-purpose computer, special purpose computer, or other programmable data-processing apparatus to produce a machine such that the instructions, which execute via the processor of the computer or other programmable data-processing apparatus, create means for implementing the functions/acts specified in the block or blocks discussed herein such as, for example, the various instructions discussed and shown with respect to the figures herein.

These computer program instructions may also be stored in a computer-readable memory that can direct a computer or other programmable data-processing apparatus to function in a particular manner such that the instructions stored in the computer-readable memory produce an article of manufacture including instruction means which implement the function/act specified in the block or blocks.

The computer program instructions may also be loaded onto a computer or other programmable data-processing apparatus to cause a series of operational steps to be performed on the computer or other programmable apparatus to produce a computer implemented process such that the instructions which execute on the computer or other programmable apparatus provide steps for implementing the functions/acts specified in the block or blocks.

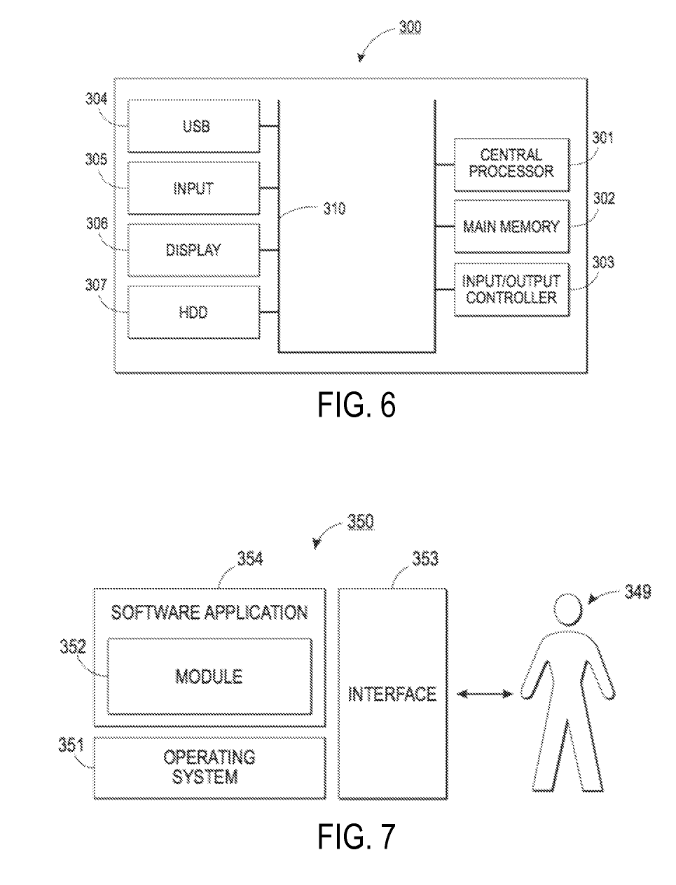

FIGS. 6-7 are provided as exemplary diagrams of data-processing environments in which embodiments of the present invention may be implemented. It should be appreciated that FIGS. 6-7 are only exemplary and are not intended to assert or imply any limitation with regard to the environments in which aspects or embodiments of the disclosed embodiments may be implemented. Many modifications to the depicted environments may be made without departing from the spirit and scope of the disclosed embodiments.

As illustrated in FIG. 6, one or more embodiments may be implemented in the context of a data-processing system 300 that can include, for example, a central processor 301 (or other processors), a main memory 302, a controller 303, and in some embodiments, a USB (Universal Serial Bus) 304 or other appropriate peripheral connection. System 300 can also include an input device 305 (e.g., a keyboard, pointing device such as a mouse, etc), a display 306, and an HDD (Hard Disk Drive) 307 (e.g., mass storage). As illustrated, the various components of data-processing system 300 can communicate electronically through a system bus 310 or similar architecture. The system bus 310 may be, for example, a subsystem that transfers data between, for example, computer components within data-processing system 300 or to and from other data-processing devices, components, computers, etc.

FIG. 7 illustrates a computer software system 350, which may be employed for directing the operation of the data-processing system 300 depicted in FIG. 6. Software application 354, stored in memory 302 and/or on HDD 307, generally can include and/or can be associated with a kernel or operating system 351 and a shell or interface 353. One or more application programs, such as module(s) 352, may be “loaded” (i.e., transferred from mass storage or HDD 307 into the main memory 302) for execution by the data-processing system 300. In the example shown in FIG. 7, module 352 can be implemented as, for example, a software module that performs the logical instructions or operations of the method, approach disclosed herein.

The data-processing system 300 can receive user commands and data through user interface 353 accessible by a user 349. These inputs may then be acted upon by the data-processing system 300 in accordance with instructions from operating system 351 and/or software application 354 and any software module(s) 352 thereof.

The discussion herein is thus intended to provide a brief, general description of suitable computing environments in which the system and method may be implemented. Although not required, the disclosed embodiments will be described in the general context of computer-executable instructions, such as program modules, being executed by a single computer. In most instances, a “module” constitutes a software application.

Generally, program modules (e.g., module 352) can include, but are not limited to, routines, subroutines, software applications, programs, objects, components, data structures, etc., that perform particular tasks or implement particular abstract data types and instructions. Moreover, those skilled in the art will appreciate that the disclosed method and system may be practiced with other computer system configurations such as, for example, hand-held devices, multi-processor systems, data networks, microprocessor-based or programmable consumer electronics, networked personal computers, minicomputers, mainframe computers, servers, and the like.

Note that the term module as utilized herein may refer to a collection of routines and data structures that perform a particular task or implements a particular abstract data type. Modules may be composed of two parts: an interface, which lists the constants, data types, variable, and routines that can be accessed by other modules or routines, and an implementation, which is typically private (accessible only to that module) and which includes source code that actually implements the routines in the module. The term module may also simply refer to an application, such as a computer program designed to assist in the performance of a specific task such as word processing, accounting, inventory management, etc.

The interface 353 (e.g., a graphical user interface) can serve to display results, whereupon a user may supply additional inputs or terminate a particular session. In some embodiments, operating system 351 and interface 353 can be implemented in the context of a “windows” system. It can be appreciated, of course, that other types of systems are possible. For example, rather than a traditional “windows” system, other operation systems such as, for example, a real time operating system (RTOS) more commonly employed in wireless systems may also be employed with respect to operating system 351 and interface 353.

FIGS. 6-7 are thus intended as examples and not as architectural limitations of disclosed embodiments. Additionally, such embodiments are not limited to any particular application or computing or data-processing environment. Instead, those skilled in the art will appreciate that the disclosed approach may be advantageously applied to a variety of systems and application software. Moreover, the disclosed embodiments can be embodied on a variety of different computing platforms, including Macintosh, Unix, Linux, and the like.

Based on the foregoing, it can be appreciated that a number of embodiments are disclosed herein. For example, in one embodiment, an apparatus for modulating body organ functioning can be implemented. Such an apparatus can include, for example, a processor and a computer-usable medium embodying computer program code, the computer-usable medium capable of communicating with the processor, the computer program code comprising instructions executable by the processor and configured for: collecting a plurality of waveform signals as analog signals comprising coded signals that are generated by at least one of axons, nerveplexus or ganglion connections of an autonomic nervous system located in a body and carried by neurons in the body, the plurality of waveform signals comprising analog signals operative in a regulation of at least on organ function of at least one body organ; converting the plurality of waveform signals from analog signals to digital signals via an analog-to-digital converter for storage in and retrieval from a memory and processing by a processor; converting the digital signals into analog signals utilizing a digital-to-analog converter after processing of the digital signals by the processor; and transmitting the analog signals to at least one body organ to regulate the at least one body organ, the analog signals comprising at least one of the plurality of waveform signals comprising a coded analog signal that regulates an organ function of the at least one body organ, the at least one of the plurality of waveform signals substantially corresponding to at least one waveform signal that is generated in a body.

In another embodiment, such instructions can be further configured for identifying a function associated at least one particular waveform signal among the plurality of waveform signals. In another embodiment, the function can be associated with a function of the at least one organ. In other embodiments, the at least one organ can be a cardiovascular organ, a respiratory organ, a digestive organ, an organ associated with appetite, etc. In some embodiments, the computer-usable medium can be a controller. In yet another embodiment, the computer-usable medium may be a USB storage drive. In still another embodiment, the computer-usable medium can be a USB portable storage drive.

In yet another embodiment, an apparatus for regulating body organ functioning in a body having a nervous system can be implemented. Such an apparatus can include, for example, a processor; and a computer-usable medium embodying computer program code, the computer-usable medium capable of communicating with the processor, the computer program code comprising instructions executable by the processor and configured for: collecting a plurality of waveforms comprising coded signals generated by at least one of axons, nerveplexus or ganglion connections of an autonomic nervous system located in the body in the body and carried by neurons in the body, the waveforms being operative in the regulation of a plurality of functions of at least one body organ; converting the plurality of waveforms into a digital signal from an analog signal via a digital-to-analog converter; converting the plurality of waveforms into an analog signal from a digital signal via an analog-to-digital converter and after processing of the plurality of waveforms by the processor; and transmitting at least one of the plurality of waveforms as the analog signal comprising a coded signal to an autonomic nervous system located in a body to regulate at least one of the plurality of functions of at least one body organ, the transmitted at least one of the plurality of waveforms substantially corresponding to at least one waveform signal that is naturally generated in the body.

In another embodiment, an apparatus for regulating body organ functioning in a body having a nervous system can be implemented. Such an apparatus can include, for example, a processor and at least one of a wire, clamp, electrode or sensor to collect a plurality of waveforms generated by at least one of axons, nerveplexus or ganglion connections of an autonomic nervous system located in the body and carried by neurons in the body, the waveforms being operative as neurosignals in the regulation of a plurality of functions of at least one body organ also located in the body; and a controller that assists in processing the waveforms by the processor, wherein the processor is capable of transmitting at least one of the plurality of waveforms from the memory through the nervous system to the at least one body organ via at least one of a wire, clamp, electrode to regulate the function of the at least one body organ, the transmitted waveform signal substantially corresponding to at least one waveform signal that is naturally generated in the body. In another embodiment, such instructions can be further configured for identifying a function associated at least one particular waveform among the plurality of waveforms.

It will be appreciated that variations of the above-disclosed and other features and functions, or alternatives thereof, may be desirably combined into many other different systems or applications. Also, that various presently unforeseen or unanticipated alternatives, modifications, variations or improvements therein may be subsequently made by those skilled in the art which are also intended to be encompassed by the following claims.

1. A apparatus for modulating body organ functioning, said apparatus comprising:
a processor; and
a computer-usable medium embodying computer program code, said computer-usable medium capable of communicating with the processor, said computer program code comprising instructions executable by said processor and configured for:
collecting a plurality of waveform signals as analog signals comprising coded signals that are generated by at least one of axons, nerveplexus or ganglion connections of an autonomic nervous system located in a body and carried by neurons in the body, said plurality of waveform signals comprising analog signals operative in a regulation of at least on organ function of at least one body organ;
converting said plurality of waveform signals from said analog signals to digital signals via an analog-to-digital converter for storage in and retrieval from a memory and processing by a processor;
converting said digital signals into analog signals utilizing a digital-to-analog converter after processing of said digital signals by said processor; and
transmitting said analog signals to at least one body organ to regulate said at least one body organ, said analog signals comprising at least one of said plurality of waveform signals comprising a coded analog signal that regulates an organ function of said at least one body organ, said at least one of said plurality of waveform signals substantially corresponding to at least one waveform signal that is generated in a body.

2. The apparatus of claim 1 wherein said instructions are further configured for identifying a function associated with at least one particular waveform signal among said plurality of waveform signals.

3. The apparatus of claim 2 wherein said function is associated with a function of said at least one organ.

4. The apparatus of claim 1 wherein said at least one organ comprises a cardiovascular organ.

5. The apparatus of claim 1 wherein said at least one organ comprises a respiratory organ.

6. The apparatus of claim 1 wherein said at least one organ comprises a digestive organ.

7. The apparatus of claim 1 wherein said at least one organ comprises an organ associated with appetite.

8. The apparatus of claim 1 wherein said computer-usable medium comprises a controller.

9. The apparatus of claim 1 wherein said computer-usable medium comprises a USB storage drive.

10. The apparatus of claim 1 wherein said computer-usable medium comprises a USB portable storage drive.

11. A apparatus for regulating body organ functioning in a body having a nervous system, said apparatus comprising:
a processor;
a computer-usable medium embodying computer program code, said computer-usable medium capable of communicating with the processor, said computer program code comprising instructions executable by said processor and configured for:
collecting a plurality of waveforms comprising coded signals generated by at least one of axons, nerveplexus or ganglion connections of an autonomic nervous system located in the body in the body and carried by neurons in the body, said waveforms being operative in the regulation of a plurality of functions of at least one body organ;
converting said plurality of waveforms into a digital signal from an analog signal via a digital-to-analog converter;
converting said plurality of waveforms into an analog signal from a digital signal via an analog-to-digital converter and after processing of said plurality of waveforms by said processor; and
transmitting at least one of said plurality of waveforms as said analog signal comprising a coded signal to an autonomic nervous system located in a body to regulate at least one of said plurality of functions of at least one body organ, said transmitted at least one of said plurality of waveforms substantially corresponding to at least one waveform signal that is naturally generated in the body.

12. The apparatus of claim 11 wherein said instructions are further configured for identifying a function associated with at least one particular waveform among said plurality of waveforms.

13. The apparatus of claim 12 wherein said function is associated with a function of said at least one body organ.

14. The apparatus of claim 1 wherein said at least one body organ comprises a cardiovascular organ, a respiratory organ, and/or a digestive organ.

15. The apparatus of claim 1 wherein said computer-usable medium comprises a controller that communicates electronically with said processor.

16. The apparatus of claim 15 wherein said computer-usable medium comprises a USB storage drive.

17. The apparatus of claim 15 wherein said computer-usable medium comprises a portable storage drive.

18. A apparatus for regulating body organ functioning in a body having a nervous system, comprising:
a processor and at least one of a wire, clamp, electrode or sensor to collect a plurality of waveforms generated by at least one of axons, nerveplexus or ganglion connections of an autonomic nervous system located in the body and carried by neurons in the body, said waveforms being operative as neurosignals in the regulation of a plurality of functions of at least one body organ also located in the body; and
a controller that assists in processing said waveforms by said processor, wherein said processor is capable of transmitting at least one of said plurality of waveforms through the nervous system to said at least one body organ via at least one of a wire, clamp, electrode to regulate the function of said at least one body organ, said transmitted waveform signal substantially corresponding to at least one waveform signal that is naturally generated in the body.

19. The apparatus of claim 18 wherein said instructions are further configured for identifying a function associated with at least one particular waveform among said plurality of waveforms.

20. The apparatus of claim 19 wherein said function is associated with a function of said at least one body organ.
